(19) United States
(12) Patent Application Publication
(10) Pub. No.: US 2025/0170043 A1
(43) Pub. Date: May 29, 2025

(54) KIT OF COSMETIC COMPOSITIONS

(71) Applicant: THE BOOTS COMPANY PLC, Nottingham (GB)

(72) Inventors: Mark Johnson, Nottingham (GB); Michael David Bell, Alfreton, Derbyshire (GB); Yegor Doush, West Bridgford, Nottingham (GB); Jake Thomas Hicks, Chilwell, Nottingham (GB); Christopher John Elms, Bottesford, Nottingham (GB)

(21) Appl. No.: 18/839,511

(22) PCT Filed: Feb. 16, 2023
(86) PCT No.: PCT/EP2023/025073

§ 371 Date: Aug. 19, 2024

(30) Foreign Application Priority Data
Feb. 18, 2022 (EP) 22020070.3

(51) Int. Cl. A61K 8/67; A61K 8/42; A61K 8/9789; A61Q 19/02; A61Q 19/08

(52) U.S. Cl. CPC A61K 8/671; A61K 8/42; A61K 8/675; A61K 8/9789; A61Q 19/02; A61Q 19/08

(57) ABSTRACT

According to the present invention there is provided a kit comprising at least 2 compositions, the first compositions comprising: a. Retinoid b. Extract of Centella asiatica and the second composition comprising ingredients selected from the group consisting of ceramide, vitamin B, extract of Ophiopogonj aponicus and mixtures thereof. According to a further aspect of the present invention, there is provided a process of application of the kit of cosmetic compositions to skin by first applying the first composition and subsequently applying the second composition in one or more application events.

### CROSS-REFERENCE TO RELATED APPLICATIONS

This application is a U.S. national stage of International Application No. PCT/EP2023/025073, filed Feb. 16, 2023, which claims priority to European Patent Application No. 22020070.3, filed Feb. 18, 2022.

### INCORPORATION BY REFERENCE OF MATERIAL SUBMITTED ELECTRONICALLY

The Sequence Listing, which is a part of the present disclosure, is submitted concurrently with the specification as an XML file. The name of the XML file containing the Sequence Listing is “70753_SeqListing.xml”, which was created on Aug. 15, 2024, and is 44,786 bytes in size. The subject matter of the Sequence Listing is incorporated herein in its entirety by reference.

### TECHNICAL FIELD

The present invention relates to a cosmetic composition kit comprising 2 cosmetics compositions. The first composition comprises a retinoid and the second composition comprise skin soothing ingredients to control and mitigate the sensitivity that can be caused by using retinoids. The present invention also describes a process for using the cosmetic composition kit.

### BACKGROUND TO THE INVENTION

The Applicant has for many years been interested in research into the ageing process of skin and in developing skincare composition to combat the natural skin-related effects of ageing. The Applicants are well known for their work with renowned universities in the field of peptide and their effect on skin, and with peptide suppliers to manufacture peptides of interest. Most recently, however, the Applicants have been focused on understanding other mechanisms for impacting and improving the appearance of skin and especially the benefits of Vitamin A and its derivatives.

It is well known in the field of cosmetic composition design and manufacture that skin is negatively impacted by endogenous and exogenous factors. Endogenous factors include hormonal fluctuations, inflammation, disease, and/or the normal ageing process. Exogenous factors affecting skin include environmental impact, for example, sunlight, wind abrasion, humidity and pollutants. The impact of these factors on the skin result in problems associated with ageing skin such as the formation of wrinkles, fine lines, age spots, enlarged pores, skin discoloration, surface roughness and loss of elasticity.

A variety of products are available over-the-counter and by prescription which are designed to treat the signs of ageing and/or promote healthier skin. There has been recent interest however in the use of vitamin A derived products in this context. Vitamin A and its derivatives are found naturally in the body and in the skin where it needs to be converted by cells into retinoic acid to be effective. Retinoic acid applied topically is therefore highly efficacious as it does not require any conversion but it has potential for adverse effects on the skin and it is therefore only available by prescription. Since therefore the larger proportion of consumers are not able to access retinoic acid containing products, cosmetic product manufacturers have instead looked toward retinyl esters and particularly retinol in over-the-counter products to treat the signs of ageing and improve the quality or appearance of skin. Retinol is the most proven and effective cosmetic alternative to retinoic acid. It binds to surface cell receptors before it is taken into skin cells and is then converted into retinoic acid which, acting via the nuclear receptors, Retinoic acid receptor (RAR) and Retinoid X receptor, (RXR) upregulates more than 500 genes involved in skin renewal, regeneration and homeostasis. This particularly benefits sun exposed skin sites such as the face, resulting in reduced signs of ageing, such as the evening of skin tone, reduced pigmentation or firmer feeling skin.

Nevertheless, high levels of retinol in products can also result in undesirable skin side effects including, but not limited to, irritation, redness, stinging, itching, skin scaling, peeling and dryness of the skin, or more severe side effects including, but not limited to, severe burning, blistering, crusting, or swelling of the skin, alterations in skin pigmentation, or aggravate eczema. Moreover, if peeling of the top layer of skin should arise, it may become more sensitive to sunlight and sun-induced damage. Finally, if irritated, the skin may be further aggravated by environmental changes and use of other cosmetic products, such as soaps or cleansers.

The present inventors have found that by offering consumers firstly a retinoid composition that produces a lower level of sensitivity on the user and secondly, a second composition designed to either smooth the skin or improve the skin barrier capability, the user perceives unexpected further reductions in age-related facial pigmentation. And additional improvements in the firmness of their skin.

It is therefore the subject of the present invention to provide a first cosmetic composition for topical application to skin comprising high potency levels of anti-ageing retinoid whilst also reducing the potentially harmful side effects described above and a second composition for use thereafter to further mitigate sensitivity, smooth the skin and improve the skin barrier function. The resulting benefit from the kit regime is an unexpected potentiation of the retinol effects; increased reductions in perceived pigmentation benefits and increased skin firmness for the kit regime versus use of the retinol on its own.

### SUMMARY OF THE INVENTION

According to the present invention there is provided a kit comprising at least 2 compositions, the first compositions comprising:
a. Retinoid
b. Extract of Centella asiatica
   and the second composition comprising ingredients selected from the group consisting of ceramide, vitamin B, extract of Ophiopogon japonicus and mixtures thereof.

According to a further aspect of the present invention, there is provided a process of application of the kit of cosmetic compositions to skin by first applying the first composition and subsequently applying the second composition in more or more applications in one or more application events.

### DETAILED DESCRIPTION OF THE INVENTION

Unless otherwise indicated, a reference to the composition of the invention is a reference to the compositions of all aspects of the invention. The present invention comprises a kit comprising a first and second composition.

### First Composition

The first composition comprises retinoid and an extract of Centella asiatica.

### Retinoid

The present invention comprises a retinoid. Said retinoid is preferably selected from the group consisting of retinol, retinol isomers, retinaldehyde (also known as retinal), retinaldehyde isomers and mixtures thereof.

The term “retinol isomers” includes the following: all-trans-retinol, 13-cis-retinol, 11-cis-retinol, 9-cis-retinol, 3,4-didehydro-retinol. Preferred isomers are all-trans-retinol, 13-cis-retinol, 3,4-didehydro-retinol, 9-cis-retinol. In one embodiment of the present invention the retinoid is selected from the group consisting of all-trans-retinol.

The term “Retinaldehyde isomers” includes the following: 11-cis retinal and all-trans-retinal. In one embodiment of the present invention the retinoid is selected from the group consisting of all-trans-retinal.

Retinoids can become unstable when exposed to light, temperature changes and oxygen, degrading to less biologically active forms, and thus in one embodiment the retinoid ingredient is preferably encapsulated. Encapsulation is designed to protect the retinoid species to prevent or at least reduce degradation, thereby maximising the bioavailability of retinoid to the skin in use. A variety of mechanisms and materials have been described in the prior art for encapsulating retinoid. Examples of encapsulating materials include polymers, surfactant based vesicles or liposomes. Preferred polymeric encapsulating materials include polysaccharide. In one embodiment, retinoid is encapsulated in a polysaccharide formulation comprising chitosan. In one embodiment, retinoid is encapsulated in a polysaccharide formulation comprising inulin. In one embodiment, retinoid is encapsulated in a polysaccharide formulation comprising hydroxy propyl cyclodextrin.

In one embodiment, the retinoid may be encapsulated with a material selected from the group consisting of sugar-based emulsifier, co-emulsifier, a positively charged molecule, solvent and mixtures thereof. One example of a preferred retinoid raw material ingredient is available commercially under the trade name InuMax sold by Evonik. Other commercially available encapsulated versions of retinol include Cyclasphere Retinol from BASF, Microsponge C116A, N720RE and N709RE from Amcol Health & Beauty Solutions and Spherulite-R10 from Givaudan. Other non-encapsulated versions of stabilised retinol are also available such as RetiSTAR stabilised retinol from BASF.

Retinoid is present in the composition of the present invention at levels of at least 0.5% by weight of the composition. In one embodiment, Retinoid is present in the compositions of the present invention at levels of at least 0.5% by weight of the composition. In another alternative embodiment, Retinoid ingredient is present in the compositions of the present invention at levels of at least 0.75% by weight of the composition. In another alternative embodiment, Retinoid ingredient is present in the compositions of the present invention at levels of at least 1% by weight of the composition.

When referencing the level of retinoid in the composition, it is expressed as a level of the active retinoid by weight of the composition and not as a percentage weight of the retinoid-containing raw material provided by a supplier.

### Centella asiatica

The composition of the present invention includes Centella asiatica, also known as Gotu kola or India Pennyworth. The present inventors have surprisingly found that at high concentrations of retinoid in the composition, Centella asiatica is more effective at reducing skin sensitivity than other known skin soothing agents, like for example bisabolol. Whilst not wishing to be bound by theory, it is believed that the success identified in respect of Centella asiatica is due to its ability to target transcription factors, such as NF-Kb, that regulate the downstream inflammatory cascade. Inhibition of NF-Kb, for example, will in turn lead to reduced production of many different mediators of inflammation such as TNF-alpha, IL-1Beta, IL-6 and Prostaglandin E. Hence Centella asiatica can promote skin soothing by impacting on a broader range of pro-inflammatory biological pathways. Bisabolol, by contrast, has been shown to be more selective impacting pro-inflammatory cytokines targets by binding with the cytokine active site.

In one embodiment, Centella asiatica is preferably extracted from the leaf of the plant. In one embodiment, Centella asiatica is characterized by the presence of biologically active triterpenes, including madecassoside contributing to the biological efficacy of the extract. In one embodiment, Centella asiatica is characterised by the presence of a polyphenolic fraction contributing to the biological efficacy of the extract. Centella asiatica provides antioxidant benefits and to promote collagen type I and III synthesis. Extract of Centella asiatica is commercially available under the trade name Centevita from Indena.

Centella asiatica may be present in the compositions of the present invention at a level of from 0.001%, 0.005% or preferably 0.01% by weight of the composition. Centella asiatics may be present in the compositions of the present invention at levels of less than 0.5%, more preferably less than 0.25% and most preferably 0.1% by weight of the composition. In one embodiment Centella asiatica may be present in the composition of the present invention at a level of from 0.001 to 0.5% by weight of the composition. In one embodiment Centella asiatica may be present in the composition of the present invention at a level of from 0.005% to 0.25% by weight of the composition. In one embodiment Centella asiatica may be present in the composition of the present invention at a level of from 0.01% to 0.1% by weight of the composition.

### Second Composition

The second composition of the present invention comprises at least one of ceramide, vitamin B and extract of Ophiopogon japonicus. In an alternative embodiment the second composition comprises at least 2 of ceramide, vitamin B and extract of Ophiopogon japonicus. In an alternative embodiment the second composition comprises ceramide, vitamin B and extract of Ophiopogon japonicus.

### Ceramide

Ceramides are a family of waxy lipid molecules. A ceramide is composed of sphingosine and a fatty acid. In an optional embodiment of the present invention, the composition comprises at least one ceramide. Whilst not be limited by theory, said ceramide may be selected from the group consisting of ceramide 1 (ceramide EOP), ceramide 2 (ceramide NG), ceramide 3 (ceramide NP) and/or ceramide 6 (ceramide AP). The above ceramides are well known in the art and defined by this nomenclature. However, for the avoidance of doubt: ceramide 1 (ceramide EOS)-comprises a sphingosine base and a non-hydroxy fatty acid chain linked to linoleic acid; ceramide 2 (ceramide NS) comprises a sphingosine base and a non-hydroxy fatty acid chain; ceramide 3 (ceramide NP) comprises a phytosphingosine base (OH group at the C4 position on the sphingosine base molecule) and a non-hydroxy fatty acid chain ceramide 6 (ceramide AP) comprises a phytosphingosine base (OH group at the C4 position on the sphingosine base molecule) and an alpha-hydroxy fatty acid chain (additional OH group on the fatty acid v.s. non-hydroxy).

In a preferred aspect of the present invention, the composition preferably comprises ceramide 3 (ceramide NP), also known as N-oloeoyl-4-hydroxysphinganine. In a further preferred but optional aspect of the present invention, the composition preferably comprises ceramide 6 (ceramide AP).

Alternatively, the composition may comprise at least two ceramides, such as ceramide 3 and ceramide 6. Alternatively, the composition may comprise at least 3 ceramides, such as ceramide 1, ceramide 3, and ceramide 6.

In one embodiment of the present invention the ceramide is Skinmimic (trade name) commercially available from Evonik. Skinmimic is an ingredient comprising a number of ceramides including Ceramide EOP, Ceramide EOS, Ceramide NP, Ceramide NS, Ceramide AP, Caprooyl Phytospingosine and Caprooyl Sphingosine.

Where present, ceramide (e.g. singularly or combined amounts of ceramides) may be present in the composition in an amount by weight of at least 0.003%, at least 0.005%, at least 0.008% or at least 0.01%; and/or the at least one ceramide (e.g. combined amounts of ceramides 1, 3 and 6) may be present in the composition in an amount by weight of 0.1% or less, 0.05% or less, 0.03% or less, or 0.01% or less.

Where present ceramide (e.g. singularly or combined amounts of ceramides) may be present in the composition in an amount by weight of from 0.003% to 0.03% or from 0.005% to 0.02%.

### Vitamin B

The second composition may preferably comprise vitamin B (e.g. vitamin B1 to vitamin B12) or a vitamin B derivative (e.g. a derivative of vitamin B1 to vitamin B12 such as a vitamin B3). In particular, the composition may comprise a vitamin B3 compound, such as niacinamide.

Vitamin B3 and niacin, are the common names for nicotinic acid. The physiologically active form of niacin is niacinamide. Niacin and niacinamide (nicotinamide or nicotinic acid amide) function in the body as a component of two coenzymes: nicotinamide adenine dinucleotide (NAD) and nicotinamide adenine dinucleotide phosphate (NADP).

The compositions used in the present invention may comprise a safe and effective amount of a (natural or synthetic) vitamin B3 compound. As used herein, “vitamin B3 compound” means a compound having the formula:

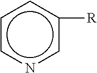

wherein R is —CONH₂ (i.e., niacinamide), —COOH (i.e., nicotinic acid) or —CH₂OH (i.e., nicotinyl alcohol); derivatives thereof; and salts of any of the foregoing.

Exemplary derivatives of the foregoing vitamin B3 compounds include nicotinic acid esters, including non-vasodilating esters of nicotinic acid, nicotinyl amino acids, nicotinyl alcohol esters of carboxylic acids, nicotinic acid N-oxide and niacinamide N-oxide.

Suitable esters of nicotinic acid include nicotinic acid esters of C1-C22, preferably C1-C16, more preferably C1-C6 alcohols. The alcohol may be straight-chain or branched chain, cyclic or acyclic, saturated or unsaturated (including aromatic), and substituted or unsubstituted. Preferred esters are those which do not yield a visible flushing response after application to the skin. Alternatively, a nicotinic acid material which does produce a flushing response can be used, if used at a lower dose to reduce the flushing effect. Non-flushing esters of nicotinic acid include tocopherol nicotinate and inositol hexanicotinate. Tocopherol nicotinate is preferred.

Other derivatives of the vitamin B3 compound are derivatives of niacinamide resulting from substitution of one or more of the amide group hydrogens. Non-limiting examples of derivatives of niacinamide useful herein include nicotinyl amino acids, derived, for example, from the reaction of an activated nicotinic acid compound (e.g., nicotinic acid azide or nicotinyl chloride) with an amino acid, and nicotinyl alcohol esters of organic carboxylic acids (e.g., C1-C18). Specific examples of such derivatives include nicotinuric acid and nicotinyl hydroxamic acid.

Exemplary nicotinyl alcohol esters include nicotinyl alcohol esters of carboxylic acids salicylic acid, acetic acid, glycolic acid, palmitic acid and the like. Other suitable vitamin B3 compounds are selected from the group consisting of 2-chloronicotinamide, 6-aminonicotinamide, 6-methylnicotinamide, n-methyl-nicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, niaprazine and mixtures thereof.

Examples of the above vitamin B3 compounds are well known in the art and are commercially available from a number of sources, e.g., the Sigma Chemical Company (St. Louis, MO); ICN Biomedicals, Inc. (Irvin, CA) and Aldrich Chemical Company (Milwaukee, WI).

The compositions of the present invention preferably comprise one or more vitamin B3 compounds. Preferred vitamin B3 compounds are niacinamide and tocopherol nicotinate. Niacinamide is more preferred.

Salts of the vitamin B3 compound are also useful herein. Non-limiting examples of salts of the vitamin B3 compound useful herein include organic or inorganic salts, such as inorganic salts with anionic inorganic species (e.g., chloride, bromide, iodide, carbonate, preferably chloride), and organic carboxylic acid salts (including mono-, di- and tri-C1-C18 carboxylic acid salts, e.g., acetate, salicylate, glycolate, lactate, malate, citrate, preferably monocarboxylic acid salts such as acetate).

The vitamin B3 compound may be included as the substantially pure material, or as an extract obtained by suitable physical and/or chemical isolation from natural sources. The vitamin B3 compound is preferably substantially pure, more preferably essentially pure.

The vitamin B or vitamin B derivative (e.g. vitamin B3 compound, such as niacinamide) may be present in the composition in an amount by weight of at least 0.01%, at least 0.05%, at least 0.1%, at least 0.5%, at least 1%, at least 2%, at least 3%, at least 4% or at least 5%; and/or the vitamin B or vitamin B derivative (e.g. vitamin B3 compound such as niacinamide) may be present in the composition in an amount by weight of 5% or less, 4% or less, 3% or less, 2% or less, 1% or less, 0.5% or less, 0.1% or less, or 0.05% or less. Where more than one vitamin B/vitamin B derivative is present, the values apply to the total amount of vitamin B or vitamin B derivative.

In particular, the vitamin B or vitamin B derivative (e.g. niacinamide) may be present in the composition in an amount by weight of from 0.01 to 5%, 0.05% to 3%, from 0.1% to 3%, 0.1% to 2%, from 0.5% to 1%, or from 0.5% to 2%.

### Extract of Ophiopogon japonicus

The second composition of the present invention may preferably comprise an extract of Ophiopogon japonicus. Preferably the raw material ingredient used in the present invention is an extract of the root of the Ophiopogon japonicus plant.

The extract is rich in purified ophiopogon tuber fructoses. The extract of Ophiopogon japonicus root comprise a steroidal glycoside known as Ophiopogonin D (OP-D) which provides anti-inflammatory benefits. Ophiopogon root also contains various additional biologically active compounds, including steroidal saponins, homoisoflavonoids, and polysaccharides, which possess anti-oxidative, anticancer, antidiabetic, immunomodulatory, anti-acute inflammatory, antitussive, and antimicrobial activities.

Extract of Ophiopogon japonicus is commercially available from Silab under the trade name Cohesium®.

Extract of Ophiopogon is present in the compositions of the present invention at a level of at least 0.1%, at least 0.5%, at least 1.0%. Extract of Ophiopogon is present in the compositions of the present invention at a level of less than 10%, less than 7.5%, less than 5.0%. Extract of Ophiopogon is present in the compositions of the present invention at a level of from 0.1% to 10.0%, 0.25% to 7.5% and preferably 0.5% to 5.0%.

### Other Components of Either the First or Second Composition

The compositions of the present invention may be aqueous or emulsion, including water-in-oil or oil-in-water emulsions. Where the composition is aqueous, it preferably comprises from 60%, more preferably more than 65% water. However, preferably the composition is an emulsion. In one embodiment the composition is an oil-in-water emulsion.

The water phase of an aqueous or emulsion preferably contains humectants which may be selected from the group consisting of diols or triols. Preferred diols are those selected from the group consisting of; pentylene glycol, caprylyl glycol, butylene glycol, di-propylene glycol, ethylhexylglycerine, propanediol, hexenediol, glycerol, butylene glycol, propylene glycol, isoprene glycol, dipropylene glycol, pentylene glycol, hexylene glycol, polypropylene glycol, butylene glycol, polyethylene glycol, sorbitol, glucitol, mannitol, hydroxypropyl sorbitol, erythritol, threitol, pentaerythritol, xylitol, and mixtures thereof. Preferred triols are those selected from the group consisting of: hexanetriol, glycerine, ethoxylated glycerin, propoxylated glycerin, and mixtures thereof.

Where the composition is an emulsion, the oil phase of an emulsion can be provided by any suitable oily component. Suitable oils for the oil phase may comprise for example: a) hydrocarbon oils, such as paraffin or mineral oils; b) waxes, such as beeswax or paraffin wax; c) natural oils, such as sunflower oil, apricot kernel oil, shea butter or jojoba oil; d) silicone oils, such as dimethicone, silicone elastomer, cyclomethicone or cetylidimethicone; e) fatty acid esters and ethers, such as isopropyl palmitate, isopropyl myristate, Caprylic/capric triglyceride, C12-15 alkyl benzoate and polypropylene glycol-15 stearyl ether; f) fatty alcohols, such as cetyl alcohol or stearyl alcohol; or g) mixtures thereof, for example, the blend of waxes available commercially under the trade name Cutina (BASF). Preferably, the emulsion comprises 0.1% to 55%, more preferably from 5% to 25%, most preferably from 10% to 20% by weight of the emulsion, of oil phase. Preferably the oil phase of the emulsion comprises oil at a level between 50% and 99.9% by weight of the oil phase. More preferably the oil phase comprises oil at a level of from 60% to 99.9%, more preferably from 70% to 99.9%, and even more preferably from 80% to 99.9% by weight of the oil phase.

Where the composition is a water-in-oil emulsion it preferably comprises an emulsifier. In a preferred embodiment, the composition comprises from 0.1% to 10% emulsifier, more preferably from 0.25% to 7.5%, still more preferably from 0.5% to 5%, emulsifier by weight of the composition. The emulsifier helps disperse and suspend the oil phase within the water phase.

### Silicone

Silicones are optional ingredients of the composition. Where present, the composition may comprise an organo polysiloxane oil. A suitable organopolysiloxane oil may be volatile, non-volatile, or a mixture of volatile and non-volatile silicones. The term “nonvolatile” as used in this context refers to those silicones that are liquid or gel under ambient conditions and have a flash point (under one atmospheric of pressure) of greater than 100° C. The term “volatile” as used in this context refers to all other silicone oils. Suitable organopolysiloxanes can be selected from a wide variety of silicones spanning a broad range of volatilities and viscosities. Examples of suitable organopolysiloxane oils include polyalkylsiloxanes, cyclic polyalkylsiloxanes, and polyalkylarylsiloxanes.

Where present, preferred for use herein are organopolysiloxanes selected from polyalkylsiloxanes, alkyl substituted dimethicones, cyclomethicones, trimethylsiloxysilicates. dimethiconols, polyalkylaryl siloxanes, and mixtures thereof. More preferred for use herein are polyalkylsiloxanes and cyclomethicones. Preferred among the polyalkylsiloxanes are dimethicones.

Optionally a suitable silicone is a silicone elastomer. Suitable for use herein are silicone elastomers which can be emulsifying or non-emulsifying crosslinked siloxane elastomers or mixtures thereof. No specific restriction exists as to the type of curable organopolysiloxane composition that can serve as starting material for the crosslinked organopolysiloxane elastomer. Examples in this respect are addition reaction-curing organopolysiloxane compositions which cure under platinum metal catalysis by the addition reaction between SiH-containing diorganopolysiloxane and organopolysiloxane having silicon-bonded vinyl groups; condensation-curing organopolysiloxane compositions which cure in the presence of an organotin compound by a dehydrogenation reaction between hydroxyl-terminated diorganopolysiloxane and SiH-containing diorganopolysiloxane and condensation-curing organopolysiloxane compositions which cure in the presence of an organotin compound or a titanate ester.

Where the composition is present as an emulsion the oil phase comprises silicone, and most preferably, a silicone elastomer. Preferably, the emulsion composition includes from 20% to 35%, by weight of the emulsion composition, of the silicone elastomer raw material.

### Saccharide Isomerate

The composition may comprise saccharide isomerate. Saccharide isomerate consists mainly of glucose, fructose mannose and galactose.

Saccharide isomerate mimics NMF (the Natural Moisturising Factor) found in human skin and is derived from plant based glucose. Saccharide isomerate is commercially available under the trademark name PENTAVITIN® from DSM Nutritional Products Ltd.

The saccharide isomerate may be present in the composition in an amount by weight of at least at least 0.05%, at least 0.1%, at least 0.2% or at least 0.3%; and/or the saccharide isomerate may be present in the composition in an amount by weight of 2% or less, 1% or less, 0.8% or less, 0.6% or less or 0.4% or less.

In particular, the saccharide isomerate may be present in the composition in an amount by weight of from 0.1% to 0.9% or from 0.2% to 0.6%.

### Antioxidant Agent

The compositions of the present invention may optionally comprise an antioxidant agent. Suitable antioxidant agents may include: a) ascorbic acid its salts, esters, glucosides and glucosamines, particularly sodium ascorbyl phosphate, magnesium ascorbyl phosphate and ascorbyl palmitate b) vitamin E (tocopherol) and its esters, particularly tocopheryl acetate, as well as Dimethyl methoxy chromanol which is a synthetic analogue of gamma tocopherol, available from Lipotec S. A. polygon Industrial Camri Ral, under the tradename Lipochroman-6 c) herbal extracts, particularly gingko biloba, such as that available under the trade name “Gingko Biloba Leaf Powder” from Univar PLC, morus alba, such as that available under the trade name “Mulberry Concentrate” from Solabia, origanum vulgare, such as that available under the trade name “Pronalen Origanum HSC” from S Black Ltd, panax ginseng, such as that available under the trade name “Panax ginseng 1.1 extract 4294”from S Black Ltd or “Phytexcell Panax ginseng” available from Croda Chemicals Ltd, birch extract such as those available from Cosmetochem (U. K.) Ltd under the trade names “Super Herbasol Extract Birch” and “HP Herbasol Betula” and those available from Blagden Chemicals under the tradenames “Phytelene of Birch” and “Aqueous Spray Dried Birch”, camellia sinensis, such as that available under the trade name “Herbal Extract Green Tea 75% Solids” from Nichimen Europe, rosmarinus officinalis, such as that available under the trade name “Pronalen Rosemary” from S. Black, Acerola cherry powder, such as that available as Acerola PE from Gee Lawson, Emlica extract sold under the tradename Emblica™ by Merck Speciality chemicals, and Grape Seed oil, such as that available from Chesham Chemicals Limited.

The source of the antioxidant activity in some of these agents is often not fully understood; for example, it is believed that the antioxidant activity of ginkgo biloba extract arises from the presence of flavonglycocides and/or terpenelactones which may be free-radical inhibitors. Birch extract may be produced by extracting the dried leaves of Betula alba with a suitable solvent. It is believed that the anti-free radical activity of birch extract arises due to the presence of flavonoids such as hyperosid, quencitrosid and/or myricetol-3digalactosid which may be free-radical inhibitors. Such products are then often sold as mixtures or solutions.

Thus the antioxidant agent may consist of a number of active ingredients which are free-radical inhibitors or may also comprise suitable diluents and/or carriers (such as when the anti-free radical agent is some of the products mentioned herein). Thus there may be some confusion as to the actual level of agent within a commercially available product. Accordingly, the amounts of antioxidant agents used in the present invention are expressed as dry weights, as understood by a man skilled in the art. The total amount of antioxidant agents present in the composition may range from 0.005% to 10% by weight, preferably 0.5% to 5%, most preferably 1% to 3.5% by weight of the composition.

Particularly preferred synergistic combinations of antioxidant agents suitable for inclusion in a skin care composition of the present invention are: panax ginseng, morus alba and magnesium ascorbyl phosphate; panax ginseng, morus alba and sodium ascorbyl phosphate; panax ginseng, morus alba and rosmarinus officinalis; panax ginseng, morus alba and origanum vulgare.

In these preferred combinations (a) the panax ginseng is preferably present in an amount of 0.005% to 0.1%, more preferably 0.01% to 0.05% by weight of the composition; (b) the morus alba is preferably present in an amount of 0.0005% to 0.01%, more preferably 0.001% to 0.005% by weight of the composition; (c) the sodium or magnesium ascorbyl phosphate is preferably present in an amount of 0.05% to 2.5%, preferably 0.1% to 2%, most preferably 0.15% to 1.5% by weight of the composition and (d) the rosmarinus officinalis or origanum vulgare is preferably present in an amount of 0.01% to 0.5%, more preferably 0.05% to 0.2% by weight of the composition.

Where an antioxidant is described as an extract of a plant, the weight of incorporation into the composition is expressed by weight of the supplier acquired raw material product and not the active antioxidant ingredient of the raw material product.

### A Monocyclic Sesquiterpene Alcohol

The composition may comprise a sesquiterpene. Sesquiterpenes are a class of terpenes that consist of three isoprene units and often have the molecular formula C₁₅H₂₄. Sesquiterpenes may be acyclic or contain rings. The present invention may additionally employ an alcohol derivative of a monocyclic (single ring) sesquiterpene. For example, the monocyclic sesquiterpene alcohol may be a-bisabolol (shown below, left) or β-bisabolol (shown below, right). Synthetic bisabolol is usually a racemic mixture: α-(±)-bisabolol

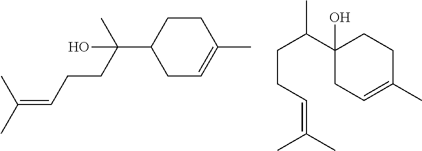

The monocyclic sesquiterpene alcohol (e.g. a-bisabolol) may be present in the composition in an amount by weight of at least 0.001%, at least 0.5%, more preferably at least 0.01%, at least 0.05%, at least 0.1%,; and/or the monocyclic sesquiterpene alcohol (e.g. α-bisabolol) may be present in the composition in an amount by weight of 2% or less, 1% or less, 0.5% or less.

In particular, the monocyclic sesquiterpene alcohol (e.g. α-bisabolol) may be present in the composition in an amount by weight of from 0.001% to 2%, from 0.01% to 1%, or from 0.05% to 0.5%.

### Emulsifiers

The composition of the present invention may comprise an emulsifier. Suitable emulsifiers include all those suitable for the purpose and known by those skilled in the art for use in skin care products. Preferably these emulsifiers have an HLB value of 5 or more, more preferably from 5 to 17, and still more preferably from 10 to 17.

Among the emulsifiers useful herein are various non-ionic and anionic emulsifying agents such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcools, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof. Nonlimiting preferred examples of these non-silicon-comprising emulsifiers include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, steareth-20, ceteareth-20, PPG-2 methyl glucose ether distearate, ceteth-10, diethanolamine cetyl phosphate, glyceryl stearate, PEG-100 stearate, and mixtures thereof.

### Peptides

The compositions of the present invention may comprise peptides. Preferably said peptides are selected from the group consisting of dipeptides, tripeptides, tetrapeptides, pentapeptides and mixtures thereof. By dipeptides, it is meant compound comprising an uninterrupted sequence of two amino acids. By tripeptides, it is meant compound comprising an uninterrupted sequence of three amino acids. By tetrapeptides, it is meant a compound comprising an uninterrupted sequence of four amino acids. By pentapeptide it is meant a compound comprising an uninterrupted sequence of five amino acids.

Optionally but preferably, the first composition comprises a dipeptide. Said dipeptide may be selected from the group consisting of acetyl dipeptide 1 cetyl ester (Tyrosine-Arginine), acetyl dipeptide 3 aminohexanoate (Alanine-Arginine), azelaoyl bisdipeptide 10 (Alanine-Histidine), coumaroyl dipeptide 3 (Alanine-Arginine), dicetyl dipeptide 9 (Glutamic acid-Lysine), dipeptide diamino butyroyl benzylamide diacetate, dipeptide 1 (Tyrosine-Arginine), dipeptide 10 (Alanine-Histidine), dipeptide 11 (Cysteine-Lysine), dipeptide 12 (Lysine-Lysine), dipeptide 14(Alanine-Threonine), (dipeptide 15 (Glycine-Glycine), dipeptide 16 (Leucine-Leucine), dipeptide 17 (Glycine-Proline), dipeptide 18 (Glycine-Histidine), dipeptide 19 (Leucine-Glutamic acid), dipeptide 2 (Valine-Tryptophan), dipeptide 20 (Lysine-Methionine), dipeptide 3 (Alanine-Arginine), dipeptide 4 (Cysteine-Glycine), dipeptide 5 (Lysine-Valine), dipeptide 6 (Proline-Hydroxyproline), dipeptide 7 (Lysine-Threonine), dipeptide 8(Alanine-Hydroxyproline), dipeptide 8 HCL (Alanine-Hydroxyproline), dipeptide 9(Glutamic acid-Lysine), hexanoyl dipeptide 3 norleucine acetate (Alanine-Arginine), methyl undecylenoyl dipeptide 16 (Leucine-Leucine), nicotinoyl dipeptide 22 (Serine-Valine), nicotinoyl dipeptide 23 (Proline-Serine), nicotinoyl dipeptide 24 (Glutamic acid-Threonine), nicotinoyl dipeptide 26 (Arginine-Valine), oleoyl dipeptide 15 (Glycine-Glycine), palmitoyl dipeptide 10 (Alanine-Histidine), palmitoyl dipeptide 13 (Tryptophan and Glutamic acid), palmitoyl dipeptide 17 (Glycine-Proline), palmitoyl dipeptide 5 diaminobutyroyl hydroxythreonine (Lysine-Valine), palmitoyl dipeptide 5 diaminohydroxybutyrate (lysine-Valine), palmitoyl dipeptide 7 (Lysine-Threonine) and mixtures thereof.

Dipeptides are preferably incorporated into the emulsion of the present invention at a level of from 0.1 to 50000 ppm, more preferably from 1 to 5000 ppm, most preferably from 10 to 500 ppm.

### Tripeptides:

The emulsions of the present invention preferably comprise a tripeptide. Said tripeptide may be naturally occurring or of synthetic origin. Suitable tripeptides include tripeptide 1, 2, 3, 4, 5, 6, 7, 8, 9, 10,11, 12, 13, 14, 15, 16, 17, 18, 19, 20, 21, 22, 23, 24, 25, 26, 27, 28,29, 30, 31, 32, 33, 34, 35, 36, 37, 38, 39, 40, 41, 42, 43, 44, 45, 46, derivatives thereof and mixtures thereof.

Particularly preferred tripeptides comprise one or more His-based tripeptides. However another suitable tripeptide may be Arg-Lys-Arg. Particularly preferred tripeptides are based on the structure Gly-His-Lys and its analogs and derivatives thereof. These are collectively known herein as GHK-tripeptides. Indeed, the preferred tripeptide in accordance with this aspect of the invention has this exact sequence of amino acids. Analogs of the preferred tripeptide useful herein include those in which one or more of the three amino acids are reorganized or rearranged within the sequence (e.g., Gly-Lys-His) and/or where no more than two amino acids are substituted (e.g., His-Ala-Orn). However, most preferably, amino acids substituted for Gly include an aliphatic side chain such as, without limitation, beta-Ala, Ala, Val, Leu, Pro, Sarcosine (Sar) and Ile. Most preferred are Ala, Leu and Ile. The most preferable amino acid substituted for Lys or His include those having a side chain that includes, predominantly, a charged nitrogen at a pH of 6, such as, without limitation, Pro, Lys, Arg, His, Desmosine and Isodesmosine. Most preferably, Lys is replaced with Orn, Arg, or Citrulline.

Derivatives are also considered to be encompassed by the term GHK-tripeptides in accordance with the present invention, (and therefore also the more generic term tripeptides). Derivatives of GHK-tripeptides in accordance with the present invention include derivatives of the substituted and rearranged tripeptides described herein. These derivatives include, inter alia, acyl-derivatives, which are tripeptides substituted with one or more straight-chain or branched-chain, long or short chain, saturated or unsaturated, substituted with a hydroxy, amino, acyl amino, sulfate or sulfide group, or unsubstituted, which can be derived from acetic acid, capric acid, lauric acid, myristic acid, octanoic acid, palmitic acid, stearic acid, behenic acid, linoleic acid, linolenic acid, lipoic acid, oleic acid, isostearic acid, elaidoic acid, 2-ethylhexaneic acid, coconut oil fatty acid, tallow fatty acid, hardened tallow fatty acid, palm kernel oil fatty acid, lanolin fatty acid and the like. Preferable examples of the acyl group include an acetyl group, a palmitoyl group, an elaidoyl group, a myristyl group, a biotinyl group and an octanoyl group. These may be substituted or unsubstituted. When substituted, they are preferably substituted with hydroxyl or sulphur compriseing groups such as, without limitation SO₃H, SH or S—S.

His-based tripeptides include at least one Histadine amine acid. The other two amino acids in the sequence may be the same or different. Thus, contemplated are, without limitation, His-Xaa-Xaa, His-Xaa-Xbb, His-Xbb-Xaa, Xbb-His-Xbb, Xbb-His-Xaa, Xaa-His-Xbb, Xaa-Xaa-His, Xaa-Xbb-His, Xbb-Xaa-His and Xbb-Xbb-His, where Xaa and Xbb are two different amino acids, although either can be His. Preferably, at least one of the other amino acids is Gly, beta-Ala, Ala, Val, Leu, Pro, Sarcosine (Sar) or Ile. Preferably, at least one of the other amino acids is Pro, Lys, Arg, His, Desmosine and Isodesmosine. Most preferably, Lys is replaced with Orn, Arg, or Citrulline. Derivatives are also considered to be encompassed by the term His-based tripeptides in accordance with the present invention, (and therefore also the more generic term tripeptides). These derivatives include, inter alia, acyl-derivatives, which are tripeptides substituted with one or more straight-chain or branched-chain, long or short chain, saturated or unsaturated substituted or unsubstituted acyl group(s) having from 1 to 29 carbon atoms. The acyl groups which can be used are the same as those described for the GHK-tripeptides.

Particularly preferred embodiments of tripeptides in accordance with the present invention include N-Acyl-Gly-His-Lys and most preferably, N-Palmitoyl-Gly-His-Lys. Preferred commercially available tripeptide and tripeptide derivative compriseing compositions include Biopeptide-CL from SEDERMA, Maxilip (R) from SEDERMA, Biobustyl (R) from SEDERMA.

The tripeptides of the present invention are preferably used in amounts that can be as little as 0.10 ppm to 10,000 ppm, preferably between 0.50 ppm to 5,000 ppm, more preferably from 1 ppm to 1000 ppm, and most preferably from 1 ppm to 500 ppm. These are again based on a % w/w basis. Thus 100,000 ppm is 10% by weight of the emulsion.

### Tetrapeptides:

The emulsions of the present invention preferably comprise a tetrapeptide. These may be one or more rigin-based tetrapeptides, one or more ALAMCAT-tetrapeptides or mixtures thereof. These tetrapeptides may be naturally occurring or of synthetic origin. Suitable tetrapeptides for use in the present composition include those selected from the group consisting of tetrapeptide 1, 2, 3, 4, 5, 6, 7, 8, 9, 10, 11, 12,13, 14, 15, 16, 17, 18, 19,20, 21, 22, 23, 24, 25,26, 27, 28, 29, 30, 34, 35, derivatives thereof and mixtures thereof.

Rigin-based tetrapeptides in accordance with the present invention are based on the structure Gly-Gln-Pro-Arg(SEQ ID NO:1) (Rigin) and include its analogs and derivatives thereof. Rigin is a preferred tetrapeptide. Analogs of the tetrapeptide rigin useful in accordance with the present invention include those in which one or more of the four amino acids are reorganized or rearranged within the sequence and/or where no more than two of the amino acids are substituted (e.g., Ala-Gln-Thr-Arg (SEQ ID NO:2. More preferably, at least one of the amino acids within the sequence is Pro or Arg and most preferably the tetrapeptide includes both Pro and Arg although their order and position may vary. The amino acid substitutions can be from amongst any amino acid as defined herein. Particularly preferred rigin-based tetrapeptides include Xaa-Xbb-Arg-Xcc, Xaa-Xbb-Xcc-Pro, Xaa-Xbb-Pro-Arg, wherein Xaa-Xbb-Pro-Xcc, Xaa-Xbb-Xcc-Arg, Xaa, Xbb and Xcc may be the same or different and selected from the following Xaa is Gly or the amino acids that may be substituted therefore, Xbb is Gln or the amino acids that may be substituted therefore and Xcc may be Pro or Arg or the amino acids substituted therefore. The most preferable amino acids substituted for Gly include an aliphatic side chain such as, without limitation, beta-Ala, Ala, Val, Leu, Pro, Sarcosine (Sar) and Ile. The most preferable amino acids substituted for Gln include a side chain that includes an amine group that is predominantly uncharged at neutral pH (pH 6-7) such as, without limitation, Asn, Lys, Orn, 5-hydroxyproline, Citrulline and Canavanine. When Arg is substituted, it is preferably replaced with an amino acid having a side chain that includes, predominantly, a charged nitrogen at a pH of 6, such as, without limitation, Pro, Lys, His, Desmosine and Isodesmosine.

Derivatives are also considered to be encompassed by the term rigin-base tetrapeptides in accordance with the present invention, (and therefore also the more generic term tetrapeptides). Derivatives include derivatives of the substituted and rearranged rigin-based tetrapeptides described herein. These derivatives include, inter alia, acyl-derivatives, which are tetrapeptides substituted with one or more straight-chain or branched-chain, long or short chain, saturated or unsaturated, substituted with a hydroxy, amino, amino acyl, sulfate or sulfide group or unsubstituted having from 1 to 29 carbon atoms. N-acyl-derivatives include those acyl groups which can be derived from acetic acid, capric acid, lauric acid, myristic acid, octanoic acid, palmitic acid, stearic acid, behenic acid, linoleic acid, linolenic acid, lipoic acid, oleic acid, isostearic acid, elaidoic acid, 2-ethylhexaneic acid, coconut oil fatty acid, tallow fatty acid, hardened tallow fatty acid, palm kernel oil fatty acid, lanolin fatty acid and the like. Preferable examples of the acyl group include an acetyl group, a palmitoyl group, an elaidoyl group, a myristyl group, a biotinyl group and an octanoyl group. These may be substituted or unsubstituted. When substituted, they are preferably substituted with hydroxyl or sulphur comprising groups such as, without limitation SO3H, SH or S—S.

ALAMCAT tetrapeptides are tetrapeptides which include at least one amino acid including an aliphatic group comprising side chain. These amino acids include, without limitation, Gly, beta-Ala, Ala, Val, Leu, Sarcosine (Sar) and Ile. These tetrapeptides also include at least one amino acid including at least one NH2 comprising side chain. These amino acids include a side chain that has an amine group that is predominantly uncharged at neutral pH (pH 6-7) such as, without limitation, Gln, Asn, Lys, Orn, 5-hydroxyproline, Citrulline and Canavanine. The ALAMCAT-tetrapeptides also include at least one amino acid having at least one side chain including at least one cationic amine (predominant species is charged such as NH3+, NH2+, etc.-basic amino acids which are positively charged at pH 6.0). These amino acids include, without limitation, Pro, Arg, Lys, His, Desmosine and Isodesmosine. The remaining amino acid can be any amino acid, but is preferably one comprising an alphatic group, pendant amino group or pendant cationic group. Derivatives are also considered to be encompassed by the term ALAMCAT-tetrapeptides in accordance with the present invention, (and therefore also the more generic term tetrapeptides). These derivatives include, inter alia, acyl-derivatives, which are tetrapeptides substituted with one or more straight-chain or branched-chain, substituted or unsubstituted long or short chain, saturated or unsaturated acyl group(s) having from 1 to 29 carbon atoms. The acyl groups which can be used are the same as those described for the rigin-based tetrapeptides.

Preferred embodiments include Peptide E, arg-ser-arg-lys(SEQ ID NO:3), N-acyl-Gly-Gln-Pro-Arg(SEQ ID NO:1) peptides, most preferably N-palmitoyl-Gly-Gln-Pro-Arg(SEQ ID NO:1).

Preferred commercially available sources of tetrapeptides include RIGIN, EYELISS, Haloxyl, and MATRIXYL 3000, which comprise between 50 to 500 ppm of palmitoyl-Gly-Gln-Pro-Arg(SEQ ID NO: 1), and other ingredients, such as peptides, chalcones and an excipient, commercially available from SEDERMA, France. Tego Pep 417 available from Evonik. These may be used to produce compositions of the present invention by adding thereto at least one tripeptide as described herein.

The tetrapeptide where present (e.g. a lipo-peptide) may have an amino acid sequence selected from the group consisting of: (i) GPXG, (ii) LSXX (iii) XXGD or (iv) QTAV(SEQ ID NO:4)

wherein for (i) G denotes the amino acid glycine and P denotes the amino acid proline (as per the internationally recognised single letter code for amino acids), and X denotes an amino acid selected from the group consisting of Lysine (K), Glutamic acid (E) and Serine(S) and mixtures thereof, wherein for (ii) L denotes the amino acid Leucine and S denotes the amino acid Serine (as per the internationally recognised single letter code for amino acids), and X denotes an amino acid selected from the group consisting of Valine (V), Aspartic acid (D), Proline (P), Glycine (G) and mixtures thereof, wherein for (iii) G denotes the amino acid Glycine and D denotes the amino acid Aspartic acid (as per the internationally recognised single letter code for amino acids), and X denotes an amino acid selected from the group consisting of Glutamic acid (E), Lysine (K), Leucine (L), Alanine (A), Isoleucine (I), Arginine (R) and mixtures thereof, and for (iv) Q denotes the amino acid Glutamine, T denotes the amino acid Threonine, A denotes the amino acid Alanine, and V denotes the amino acid Valine.

In another embodiment, the peptide (e.g. a lipo-peptide) has an amino acid sequence selected from the group consisting of: (i) Y-GPXG-Z, (ii) Y-LSXX-Z (iii) Y-XXGD-Z or (iv) Y-QTAV-Z (SEQ ID NO:4) wherein for (i) G denotes the amino acid glycine and P denotes the amino acid proline (as per the internationally recognised single letter code for amino acids), and X denotes an amino acid selected from the group consisting of Lysine (K), Glutamic acid (E) and Serine(S) and mixtures thereof, wherein for (ii) L denotes the amino acid Leucine and S denotes the amino acid Serine (as per the internationally recognised single letter code for amino acids), and X denotes an amino acid selected from the group consisting of Valine (V), Aspartic acid (D), Proline (P), Glycine (G) and mixtures thereof, wherein for (iii) G denotes the amino acid Glycine and D denotes the amino acid Aspartic acid (as per the internationally recognised single letter code for amino acids), and X denotes an amino acid selected from the group consisting of Glutamic acid (E), Lysine (K), Leucine (L), Alanine (A), Isoleucine (I), Arginine (R) and mixtures thereof, wherein for (iv) Q denotes the amino acid Glutamine, T denotes the amino acid Threonine, A denotes the amino acid Alanine, and V denotes the amino acid Valine, and wherein at the N-terminal end, Y is selected from the group consisting of H, —CO—R¹, —SO₂—R¹ or a biotinyl group, at the C-terminal end, Z is selected from the group consisting of OH, O R¹, NHR¹ or NR¹R².

In one embodiment, R¹ and R² are independently selected from the group consisting of alkyl, aryl, aralkyl, alkylaryl, alkoxy, saccharide and aryloxy group, which may be linear, branched, cyclical, polycyclic, unsaturated, hydroxylates, carbonylated, phosphorylated and/or sulphurous, said groups comprising from 1 to 24 carbon atoms and being capable of including one or more heteroatoms O, S and/or N.

In a preferred embodiment of the present invention the tetrapeptide is modified at the N-terminal and/or the C-terminal end.

In a preferred embodiment the tetrapeptides of the present invention is selected from the group consisting of: Y-EKGD-Z(SEQ ID NO:5), Y-ELGD-Z(SEQ ID NO:6), Y-EAGD-Z(SEQ ID NO:7), Y-EIGD-Z(SEQ ID NO:8), Y-ERGD-Z(SEQ ID NO:9), Y-KEGD-Z(SEQ ID NO: 10), Y-KLGD-Z(SEQ ID NO:11), Y-KAGD-Z(SEQ ID NO:12), Y-KIGD-Z(SEQ ID NO:13), Y-KRGD-Z(SEQ ID NO:14), Y-LEGD-Z(SEQ ID NO:15), Y-LKGD-Z (SEQ ID NO:16), Y-LAGD-Z (SEQ ID NO:17), Y-LIGD-Z(SEQ ID NO:18), Y-LRGD-Z (SEQ ID NO:19), Y-IEGD-Z (SEQ ID NO:20), Y-IKGD-Z(SEQ ID NO:21), Y-ILGD-Z(SEQ ID NO:22), Y-IAGD-Z(SEQ ID NO:23), Y-IRGD-Z(SEQ ID NO:24), Y-REGD-Z(SEQ ID NO:25), Y-RKGD-Z(SEQ ID NO:26), Y-RLGD-Z(SEQ ID NO:27), Y-RAGD-Z(SEQ ID NO:28), Y-RIGD-Z(SEQ ID NO:29), Y-AEGD-Z(SEQ ID NO:30), Y-AKGD-Z(SEQ ID NO:31), Y-ALGD-Z(SEQ ID NO:32), Y-AIGD-Z(SEQ ID NO:33) and Y-ARGD-Z(SEQ ID NO:34). In another embodiment, the tetrapeptides is selected from the group consisting of Y-EKGD-Z(SEQ ID NO:5), Y-LKGD-Z(SEQ ID NO:16), Y-IRGD-Z(SEQ ID NO:24) and Y-AKGD-Z(SEQ ID NO:31). In another embodiment the tetrapeptide is Y-EKGD-Z(SEQ ID NO:5). In another embodiment the tetrapeptide is Y-LKGD-Z(SEQ ID NO:16). In another embodiment the tetrapeptide is Y-IRGD-Z(SEQ ID NO:24). In another embodiment the tetrapeptide is Y-AKGD-Z(SEQ ID NO:31).

In a preferred embodiment the tetrapeptide combination of the present invention, tetrapeptide a) is selected from the group consisting of: Y-LSVD-Z(SEQ ID NO:35), Y-LSVP-Z, (SEQ ID NO:36) Y-LSVG-Z(SEQ ID NO:37), Y-LSDV-Z(SEQ ID NO:38), Y-LSDP-Z(SEQ ID NO:39), Y-LSDG-Z(SEQ ID NO:40), Y-LSPV-Z(SEQ ID NO:41), Y-LSPD-Z(SEQ ID NO:42), Y-LSPG-Z(SEQ ID NO:43), Y-LSGV-Z(SEQ ID NO:44), Y-LSGD-Z(SEQ ID NO:45) and Y-LSGP-Z(SEQ ID NO:46). In another embodiment, the tetrapeptides is selected from the group consisting of Y-LSVD-Z(SEQ ID NO:35), Y-LSPG-Z(SEQ ID NO:43) and Y-LSPD-Z(SEQ ID NO:42). In another embodiment the tetrapeptide is Pal-LSVD-OH(SEQ ID NO:35). In another embodiment the tetrapeptide is Pal-LSPG-OH(SEQ ID NO:43). In another embodiment the tetrapeptide is Pal-LSPD-OH(SEQ ID NO:42).

In a further preferred embodiment of the present invention, the tetrapeptide is Y-GPKG-Z(SEQ ID NO:47). In a further preferred embodiment of the present invention the tetrapeptide is Y-GPEG-Z(SEQ ID NO:48). In a further preferred embodiment of the present invention the tetrapeptide is Y-GPSG-Z(SEQ ID NO:49).

The tetrapeptides of the present invention are preferably used in amounts from 0.1 ppm (0.00001% w/w also referred to herein as “weight percent”, “weight %” or simply by weight) to 10,000 ppm (0.5% w/w), preferably from 0.5 ppm to 1000 ppm (0.05% w/w), and most preferably from 1 ppm to 500 ppm by weight of the composition.

The combination of tripeptides and tetrapeptides, is particularly preferred. The preferred ratio of tetrapeptide to tripeptide, or indeed the ratio of molecules having four amino acids to those having three amino acids can range from 100:1 to 1:100; more preferably from 50:1 to 1:50, even more preferably from 30:1 to 1:30 and even more preferably between 10:1 to 1:10. Most preferably, the ratio of tetrapeptide to tripeptide ranges from between 3:1 to 1:3. These ratios are on a weight basis (% w/w-e.g. mg of pure peptide per Kilogram in the final formulation). In a particularly preferred embodiment, the amount of tripeptide used is greater than the amount of tetrapeptide used when considered in terms of their amounts in parts per million, again based on overall weight of the composition. In a particularly preferred embodiment, the emulsion of the present invention comprise a tetrapeptide of the sequence Gly-Gln-Pro-Arg(SEQ ID NO:1), its analogs and derivatives in combination with one or more tripeptide of the sequences Gly-His-Lys, its analogs and derivatives.

### Pentapeptides

The compositions of the present invention may optionally comprise a pentapeptide, derivatives of pentapeptides, and mixtures thereof. As used herein, “pentapeptides” refers to both the naturally occurring pentapeptides and synthesized pentapeptides. Also useful herein are naturally occurring and commercially available compositions that comprise pentapeptides. Suitable pentapeptides are those selected from the group consisting of pentapeptide 1, 4, 5, 6, 7, 8, 9, 10, 11, 12, 13, 14, 15, 16, 17, 18, 19, 21, 22, 23, 24, 25, 26, 28, 29, 30, 31, 33, 34, 35, 36, 38, 39, derivatives thereof and mixtures thereof.

Suitable pentapeptides for use herein are the pentapeptide, lys-thr-thr-lys-ser(SEQ ID NO:50), Arg-asp-lys-tyr-val (SEQ ID NO:51) (pentapeptide-1) and derivatives thereof. A preferred commercially available pentapeptide derivative-comprising composition is Matrixyl which comprises 100 ppm of palmitoyl-lys-thr-thr-lys-ser(SEQ ID NO:50) and is commercially available from Sederma, France.

The pentapeptides and/or pentapeptide derivatives where present are preferably included in emulsion at amounts of from 0.01% to 20%, more preferably from 0.05% to 15%, and even more preferably from 0.1% to 10%, by weight of the composition.

### Matrix Metalloproteinase Inhibitors (MMPi)

The composition of the present invention may comprise a matrix metalloproteinase inhibitor. The term “matrix metalloproteinase inhibitor” relates to all molecule and/or plant or bacterial extracts having an inhibitory activity on at least one of the matrix metalloproteinases expressed or synthetized by or in the skin. The family of the matrix metalloproteinases is formed of several well-defined groups on the basis of their resemblance regarding structure and substrate specificity (Woessner J. F., Faseb Journal, vol. 5,1991, 2145). Among these groups, there are collagenases able to degrade fibrillar collagens (MMP-1 or interstitial collagenase, MMP-8 or neutrophil collagenase, MMP-13 or collagenase 3, MMP-18 or collagenase 4), gelatinases degrading type IV collagen or other denatured collagen form (MMP-2 or A gelatinase (72 kDa), MMP-9 or B gelatinase (92 kDa)), stromelysins (MMP-3 or stromelysin 1, MMP-10 or stromelysin 2, MMP-1l or stromelysin 3) whose broad spectrum of activity targets proteins of the extracellular matrix such as glycoproteins (fibronectin, laminin), proteoglycanes etc., matrilysin (MMP-7), metalloelastase (MMP-12) or metalloproteinases (MMP-14, MMP-15, MMP-16 and MMP-17). Metalloproteinases (MMPs) are proteases that use a metal, (mostly zinc) coordinated to 3 cystein residues and to a methionine in their active site, that degrade macromolecular components of the extracellular matrix and of basal layers at neutral pH (collagen, elastin, etc . . . ). This group of enzymes is inactivated by metal chelators. The principal activity regulators of MMPs are the tissue inhibitors of metalloproteinases or TIMPs such TIMP-I, TIMP-2, TIMP-3 and TIMP-4 (Woessner J. F., Faseb Journal, 1991). Furthermore, the MMPs expression is also regulated by growth factors, cytokins, oncogens products (ras, jun), or also matrix constituents.

The term “matrix metalloproteinase inhibitors” according to the present invention means all molecules able to reduce the MMPs activity regarding the gene expression (transcription and translation) or regarding the activation of the zymogen form of MMPs, or else regarding the local control of active forms. Furthermore, the metalloproteinase inhibitors according to the present invention can also be MMP-1 inhibitors of natural or synthetic origin. The terms “natural origin” or “synthetic origin” mean both a metalloproteinase inhibitor at a pure state or in solution at different concentrations, but natural origin termed inhibitors are obtained by different extraction methods from a natural element (for example lycopene from a tomato) whereas the inhibitors of synthetic origin are all obtained via chemical synthesis

Preferred MMPi are selected from the group consisting of retinoid, N-acetyl cysteine, glutathione, 2-furildioxime, vitamin C, flavones, isoflavones, hydrolysed rice protein, alfalfa extract, white lupin, zizyphus jujube extract, dihydroxy methyl chromone, kudzu extract, vitis vinifera extract, Oenothera biennis extract Anogeissus leiocarpus extract and mixtures thereof.

Where present MMPi are present at a level of from 0.01% to 10%, more preferably 0.1% to 5% and most preferably from 1% to 2.5% by weight of the composition.

### Skin Conditioning Agent

The compositions of the present invention may optionally comprise a skin conditioning agent. Said skin conditioning agents may preferably be selected from the group consisting of humectants, emollients, moisturisers, or mixtures thereof. Where present, they are preferably present at a level of from 0.01% to 20%, more preferably from 0.1% to 10%, most preferably from 0.5% to 7% by weight of the emulsion.

Preferred skin conditioning agents are selected from the group consisting of guanidine, urea, glycolic acid and glycolate salts, salicylic acid, lactic acid and lactate salts, aloe vera, shea butter, polyhydroxy alcohols, such as sorbitol, mannitol, xylitol, erythritol, glycerol, hexanetriol, butanitriol, (di) propylene glycol, butylene glycol, hexylene glycol, polyethylene glycol, sugars (e.g. fructose, glucose, xylose, honey, mannose, xylose), gluconodeltalactone, and starches and their derivatives, pyrrolidone, carboxylic acid, hyaluronic acid and salts thereof, lactamide monoethanolamine, acetamide monoethanolamine, panthenol, allantoin and mixtures thereof.

The composition may comprise hyaluronic acid or a salt thereof, such as sodium hyaluronate or potassium hyaluronate. The composition preferably comprises sodium hyaluronate.

More preferably said skin conditioning agent is selected from glycerine, arabinoglactan, butylene glycol, hyaluronic acid, shea butter, propylene glycol, ethylhexyl glycerin and hyaluronate.

### Vitamins

The compositions of the present invention may comprise one or more additional vitamins. The compositions may comprise ascorbates, for example vitamin C, vitamin C derivatives, ascorbic acid, ascorbyl glucoside, ascorbyl palmitate, magnesium ascorbyl phosphate, sodium ascorbyl phosphate. The compositions may comprise vitamin K, vitamin K derivatives, vitamin H vitamin D, vitamin D derivatives, vitamin E, vitamin E derivatives such as tocopherol and tocopheryl acetate, and provitamins thereof, such as panthenol and mixtures thereof. The vitamin compounds may be included as the substantially pure material, or as an extract obtained by suitable physical and/or chemical isolation from natural (e. g., plant) sources. In one embodiment, when vitamin compounds are present in the compositions of the instant invention, the compositions comprise from about 0.0001% to 50%, more preferably from 0.001% to 10%, still more preferably from 0.01% to 8%, and still more preferably from 0.1% to 5%, by weight of the composition, of the pure vitamin compound.

### Other Optional Ingredients

The compositions of the present invention may also optionally comprise one or more of the following optional ingredients. Preservatives may be added to the composition such as 2-bromo2-nitropropane-1,3-diol (bronopol, which is available commercially under the trade name Myacide RTM), benzyl alcohol, diazolidinyl urea, imidazolidinyl urea, methyl paraben, phenoxy ethanol, ethyl paraben, propyl paraben, sodium methyl paraben, sodium dehydroacetate, polyhexamethylenebiguanide hydrochloride, isothiazolone and sodium propyl paraben, suitably in an amount of from 0.01% to 10% by weight of the composition.

Thickeners, viscosity modifying agents and/or gelling agents may be added to the composition, such as acrylic acid polymers e. g. available commercially under the trade name Carbopol, Pemulen or Ultrez (Lubrizol) or modified cellloses e. g. hydroxyethylcellulose available commercially under the trade name Natrosol (Hercules) or hydroxypropylmethyl cellulose, amine oxides, block polymers of ethylene oxide and propylene oxide (for example, those available from BASF Wyandotte under the trade name Pluronic“RTM), PVM, MA, or a decadiene crosspolymer (available under the trade name Stabilez 60), ethoxylated fatty alcohols, salt (magnesium chloride, sodium chloride), Aristoflex AVC (Clariant), phthalic acid amide, xanthan gum, sodium polyacrylate, polyvinyl alcools, fatty alcools and alkyl galactmanans available under the trade name N-Hance from Hercules, suitably in an amount of from 0.5% to 10% by weight of the composition.

Sequestering agents may be added to the composition, such as ethylenediamine tetraacetic acid and salts thereof, suitably in an amount of from 0.005% to 0.5% by weight of the composition.

The composition may also include waxes such as cocoa butter suitably in an amount of from 1% to 99% by weight of the composition.

The composition may also comprise suitable, cosmetically acceptable diluents, carriers and/or propellants such as dimethyl ether.

The composition may also include pearlising agents such as stearic monoethanolamide and/or mica, suitably in an amount of from 0.01% to 10% by weight of the composition.

Perfumes may be added suitably in an amount of from 0.01% to 2% by weight of the composition, as may water soluble dyes such as tartrazine, suitably in an amount of from a trace amount (such as 1×10-5%) to 0.1% by weight of the composition.

The composition may also include pH adjusting agents such as sodium hydroxide, aminomethyl propanol, triethanolamine, suitably in an amount of from 0.01% to 10% by weight of the composition. The composition may be buffered by means well known in the art, for example by use of buffer systems comprising succinic acid, citric acid, lactic acid, and acceptable salts thereof, phosphoric acid, mono-or disodium phosphate and sodium carbonate. Suitably, the composition may have a pH between 3 and 10, preferably between 4 and 8.

### Confidential User Trial

The compositions of the present invention were used in confidential user trial with participants. Three parallel group 4-week home user trials were performed on a total of 450 women (150 participants per group) aged 30-70 years old. The participants were selected by virtue of their keen interest in skin care and a desire for younger looking skin which included an improvement in the evenness of their facial skin tone and a reduction in pigmentation. Group 1 used a high concentration retinol containing formulation (composition A) as per example 1, applying it at night twice a week on non-consecutive nights to start with and ramping up usage to nightly use after 4 weeks. Group 2 used a different high retinol containing formulation with soothing barrier ingredients included within the composition (composition B) as per example 1. Group 2 were given the same usage instructions as Group 1. Finally, Group 3 used a kit of Prototype A in addition to a soothing barrier formulation as per example 2 and were instructed to apply the second composition nightly for 4 weeks in replacement of a regular night cream. The soothing barrier formulation was always applied after the high concentration retinol formulation. The above is summarised in table 1.

TABLE 1
summary of usage trial

| | | Number of | | |
|---|---|---|---|---|
| | | women | Average | |
| | Products | who | age of | |
| Group | assessed | participated | women | Usage |
| 1 | High | 150 | 52 | Twice a week, |
| | concentration | | | night only to |
| | retinol | | | start ramping to |
| | composition A | | | nightly after |
| | | | | 4 weeks. |
| 2 | High | 150 | 49 | Twice a week, |
| | concentration | | | night only to |
| | retinol | | | start ramping |
| | composition B | | | to nightly after |
| | | | | 4 weeks. |
| 3 | Kit of high | 150 | 54 | High |
| | concentration | | | concentration |
| | retinol | | | retinol using |
| | (composition A) + | | | composition A |
| | soothing barrier | | | as above. |
| | regime | | | Soothing barrier |
| | | | | applied nightly. |

The participants of Groups 1, 2 and 3 used the products and kit for 4 successive weeks and were asked to agree or disagree with statements about their skin e.g. My skin pigmentation has reduced. The participants were requested to choose from a 4-point scale, as follows:
1. Strongly disagree
2. Slightly disagree
3. Slightly agree
4. Strongly agree

Strongly agree and slightly agree were regarded as positive responses. The number of participants who scored the statement positively (i.e. gave the response strongly agree and slightly agree) was tabulated as in tables 2a and b below.

TABLE 2a
Pigmentation results of the confidential user trial

| Statement: My skin | | | | |
|---|---|---|---|---|
| pigmentation has reduced | 1 week | 2 weeks | 3 weeks | 4 weeks |
| Composition A (High retinol | 38% | 51% | 65% | 68% |
| containing formulation) | | | | |
| Composition B (High retinol | 35% | 53% | 66% | 70% |
| containing formulation | | | | |
| with soothing barrier | | | | |
| ingredients included) | | | | |
| Kit composition A + Soothing | 45% | 56% | 74% | 81% |
| barrier formulation regime | | | | |

TABLE 2b
Skin firmness results of the confidential user trial

| Statement: My skin feels firmer | 1 week | 2 weeks | 3 weeks | 4 weeks |
|---|---|---|---|---|
| Composition A (High retinol | 28% | 57% | 62% | 72% |
| containing formulation) | | | | |
| Composition B (High retinol | 35% | 62% | 65% | 73% |
| containing formulation | | | | |
| with soothing barrier | | | | |
| ingredients included) | | | | |
| Kit composition A + Soothing | 63% | 73% | 77% | 80% |
| barrier formulation regime | | | | |

Tolerance and skin sensitivity were also monitored during the trial using the classifications below created with support from a dermatologist. Participants were asked to classify the tolerance and sensitivity of their skin using the table below.

TABLE 3
Skin sensitivity classification

TOLERANT
No reactions or unpleasant sensations felt or seen
MILD
Tingling, stinging, tightness, blemishes/spots,
peeling and dryness without soreness, slight
redness, slight feeling of heat/burning.
MODERATE
Red, angry and sore to the touch, blind pimples
beneath the surface of the skin, large area of
dryness, rash.
SEVERE
Red, angry and sore without touching, eczema-like
and persistent. Reactions can include broken
skin, blistering, extended rash.

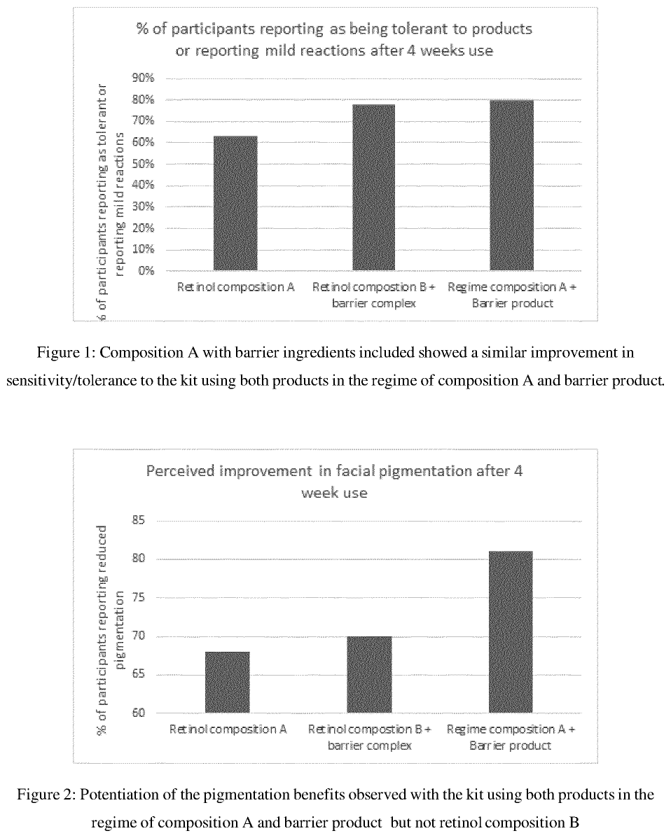

The results of this study demonstrated that skin tolerance could be improved with the inclusion of barrier enhancing ingredients in the single high concentration retinol product (Group 2: composition B) or with them included into a kit of separate and additional barrier product for use together with the retinol product (Group 3: Kit of composition A+Barrier product) in a regime. Levels of tolerance improvement in group 2 and 3 were similar (see FIG. 1). However, it was surprising that the kit of compositions (Group 3: Kit of composition A+Barrier product) could potentiate the pigmentation (shown in FIG. 2) and firming efficacy of the retinol with more participants observing improvements on the kit versus on the single retinol products.

### Example Compositions

The below provides non-limiting examples of the first and second compositions according to the present invention

### REPRESENTATIVE EXAMPLES OF THE FIRST COMPOSITION

### Example 1: First Composition, Composition A (High Retinol Containing Formulation)—Oil in Water Lotion

| Material | % w/w |
|---|---|
| Dimethicone | 5.50 |
| Water | 72.52 |
| Glycerin | 5.00 |
| Dimethicone crosspolymer & | 1.00 |
| Dimethicone | |
| Phenoxyethanol & Caprylyl glycol & | 0.80 |
| Ethylhexylglycerin | |
| Glyceryl stearate & PEG-100 stearate | 2.00 |
| Cetearyl alcohol | 2.00 |
| Sodium polyacrylate | 0.60 |
| Xanthan gum | 0.10 |
| Tetrasodium EDTA | 0.05 |
| Retinol complex (Retinol encapsulated | 9.38 |
| with inulin lauryl carbamate, | |
| Behentrimonium chloride) | |
| Acetyl dipeptide-1 cetyl ester | 1.00 |
| Centella asiatica leaf extract | 0.05 |

### Method of Manufacture

1. To water add glycerin and dissolve tetrasodium EDTA.
2. Using homogenisation sprinkle in xanthan gum and continue to homogenise for 5 minutes or until hydrated.
3. Heat water phase to 70-75° C.
4. In a separate vessel weigh out oil phase and heat to 70-75° C. (Dimethicone, cetearyl alcohol, glyceryl stearate & PEG-100 stearate) When at temperature stir in the sodium polyacrylate.

5. With both phases at 70-75° C. add the oil phase to the water phase and homogenise for 2 minutes.
6. Add dimethicone crosspolymer & dimethicone and homogenise for 2 minutes.
   1. Cool to room temperature.
   2. Stir in Phenoxyethanol & Caprylyl glycol & Ethylhexylglycerin, Acetyl dipeptide-1 cetyl ester and Retinol complex.
   3. Dissolve Centella asiatica leaf extract in a small amount of water and stir into the bulk.
   4. Make to weight with water and stir smooth.

### Example 2—First Composition-Oil in Water Serum

| Material | % w/w |
|---|---|
| Dimethicone & Dimethiconol | 1.50 |
| Dimethicone | 2.00 |
| Water | 75.82 |
| Glycerin | 5.00 |
| Caprylic/capric triglyceride | 2.00 |
| Phenoxyethanol & Caprylyl glycol & | 0.80 |
| Ethylhexylglycerin | |
| Acrylates.C10-30 alkyl acrylate | 0.20 |
| crosspolymer | |
| Butylene glycol | 2.00 |
| Potassium hydroxide | 0.10 |
| Xanthan gum | 0.10 |
| Tetrasodium EDTA | 0.05 |
| Retinol complex (Retinol encapsulated | 9.38 |
| with inulin lauryl carbamate, | |
| Behentrimonium chloride) | |
| Acetyl dipeptide-1 cetyl ester | 1.00 |
| Centella asiatica leaf extract | 0.05 |

### Method of Manufacture

1. To water add glycerine, butylene glycol and dissolve tetrasodium EDTA.
2. Using homogenisation sprinkle in xanthan gum and Acrylates.C10-30 alkyl acrylate crosspolymer continue to homogenise for 5 minutes or until hydrated.
3. In a separate vessel weigh out oil phase (Dimethicone and caprylic/capric triglyceride) and stir until solids dissolved
4. Add the oil phase to the water phase and homogenise for 2 minutes.
1. Add dimethicone & dimethiconol and homogenise for 2 minutes.
2. Add the potassium hydroxide and homogenise for 2 minutes.
3. Stir in Phenoxyethanol & Caprylyl glycol & Ethylhexylglycerin, Acetyl dipeptide-1 cetyl ester and Retinol complex.
4. Dissolve Centella asiatica leaf extract in a small amount of water and stir into the bulk.
5. Make to weight with water and stir smooth.

### Example 3—First Composition, Composition B—(High Retinol Containing Composition Soothing Barrier Ingredients)-Oil in Water lotion

| Material | % w/w |
|---|---|
| Dimethicone | 5.50 |
| Water | 70.82 |
| Glycerin | 5.00 |
| Dimethicone crosspolymer & | 1.00 |
| Dimethicone | |
| Phenoxyethanol & Caprylyl glycol & | 0.80 |
| Ethylhexylglycerin | |
| Glyceryl stearate & PEG-100 stearate | 2.00 |
| Cetearyl alcohol | 2.00 |
| Sodium polyacrylate | 0.60 |
| Xanthan gum | 0.10 |
| Tetrasodium EDTA | 0.05 |
| Retinol complex (Retinol encapsulated | 9.38 |
| with inulin lauryl carbamate, | |
| Behentrimonium chloride) | |
| Niacinamide | 1.00 |
| Ceramide complex (blend of Cholesterol | 1.00 |
| & Ceramide AP & Ceramide EOP & | |
| Ceramide NP & Phytosphingosine) | |
| Bisabolol | 0.20 |
| Panthenol | 0.50 |
| Centella asiatica leaf extract | 0.05 |
| Dipotassium glycyrrhizate | 0.5 |

### Method of Manufacture

1. To water add glycerin and dissolve tetrasodium EDTA.
2. Using homogenisation sprinkle in xanthan gum and continue to homogenise for 5 minutes or until hydrated.
3. Heat water phase to 70-75° C.
4. In a separate vessel weigh out oil phase and heat to 70-75° C. (Dimethicone, cetearyl alcohol, Bisabolol, glyceryl stearate & PEG-100 stearate) When at temperature stir in the sodium polyacrylate.
5. With both phases at 70-75° C. add the oil phase to the water phase and homogenise for 2 minutes.
6. Add dimethicone crosspolymer & dimethicone and homogenise for 2 minutes.
7. Cool to room temperature.
8. Stir in Phenoxyethanol & Caprylyl glycol & Ethylhexylglycerin, Ceramide complex, Panthenol and Retinol complex.
9. Dissolve Centella asiatica leaf extract, Dipotassium glycyrrhizate and niacinamide separately in a small amount of water and stir into the bulk.
10. Make to weight with water and stir smooth.

### REPRESENTATIVE EXAMPLES OF THE SECOND COMPOSITION

### Example 4—Second Composition Barrier Product—Oil in Water Cream

| Material | % w/w |
|---|---|
| Dimethicone | 1.50 |
| Butyrospermum Parkii (Shea) Butter | 3.00 |
| Simmondsia Chinensis (Jojoba) Seed Oil | 1.00 |
| C12-15 alkyl benzoate | 4.00 |
| Water | 73.20 |
| Glycerin | 6.00 |
| Glyceryl stearate & PEG-100 stearate | 2.00 |
| Phenoxyethanol & Caprylyl glycol & | 0.80 |
| Ethylhexylglycerin | |
| Potassium cetyl phosphate | 0.20 |
| Cetearyl alcohol | 2.00 |
| Sodium polyacrylate | 0.60 |
| Tetrasodium EDTA | 0.05 |
| Ceramide complex (blend of Cholesterol | 1.00 |
| & Ceramide AP & Ceramide EOP & | |
| Ceramide NP & Phytosphingosine) | |
| Panthenol | 0.50 |
| Bisabolol | 0.10 |
| Niacinamide | 1.00 |
| Ophiopogon japonicus root extract & | 2.50 |
| Water | |
| Centella asiatica leaf extract | 0.05 |
| Dipotassium glycyrrhizate | 0.50 |

### Method of Manufacture

1. To water add glycerin and dissolve tetrasodium EDTA.
2. Heat water phase to 70-75° C.
3. In a separate vessel weigh out oil phase and heat to 70-75° C. (Dimethicone, cetearyl alcohol, Butyrospermum Parkii (Shea) Butter, Simmondsia Chinensis (Jojoba) Seed Oil, C12-15 alkyl benzoate, potassium cetyl phosphate, Bisabolol, glyceryl stearate & PEG-100 stearate) When at temperature stir in the sodium polyacrylate.
4. With both phases at 70-75° C. add the oil phase to the water phase and homogenise for 2 minutes.
5. Cool to room temperature.
6. Stir in Phenoxyethanol & Caprylyl glycol & Ethylhexylglycerin, Ceramide complex, Panthenol, Ophiopogon japonicus root extract & Water,
7. Dissolve Centella asiatica leaf extract, Niacinamide and Dipotassium glycyrrhizate separately in a small amount of water and stir into the bulk.
8. Make to weight with water and stir smooth.

### Example 5—Second Composition Barrier Product—Water in Oil Cream

| Material | % w/w |
|---|---|
| Dimethicone | 0.50 |
| Butyrospermum Parkii (Shea) Butter | 3.00 |
| Simmondsia Chinensis (Jojoba) Seed Oil | 1.00 |
| C12-15 alkyl benzoate | 6.00 |
| Water | 72.00 |
| Glycerin | 6.00 |
| Magnesium sulfate | 1.00 |
| Cetyl PEG/PPG-10/1 dimethicone & | 4.00 |
| Polyglyceryl-4 isostearate & Hexyl | |
| laurate | |
| Phenoxyethanol & Caprylyl glycol & | 0.80 |
| Ethylhexylglycerin | |
| Tetrasodium EDTA | 0.05 |
| Ceramide complex (blend of Cholesterol | 1.00 |
| & Ceramide AP & Ceramide EOP & | |
| Ceramide NP & Phytosphingosine) | |
| Panthenol | 0.50 |
| Bisabolol | 0.10 |
| Niacinamide | 1.00 |
| Ophiopogon japonicus root extract & | 2.50 |
| Water | |
| Centella asiatica leaf extract | 0.05 |
| Dipotassium glycyrrhizate | 0.50 |

### Method of Manufacture

To water add glycerine, Ophiopogon japonicus root extract & Water, panthenol, Ceramide blend and dissolve tetrasodium EDTA, magnesium sulfate Centella asiatica leaf extract, Dipotassium glycyrrhizate,
1. Heat water phase to 45-50° C.
2. In a separate vessel weigh out oil phase and heat to 45-50° C. (Dimethicone, Butyrospermum Parkii (Shea) Butter, Simmondsia Chinensis (Jojoba) Seed Oil, C12-15 alkyl benzoate, Bisabolol, Cetyl PEG/PPG-10/1 dimethicone & Polyglyceryl-4 isostearate & Hexyl laurate) When at temperature stir in the sodium polyacrylate.
3. With both phases at 70-75° C. add the water phase to the oil phase slowly until an emulsion is formed.
4. Cool to room temperature.
5. Stir in Phenoxyethanol & Caprylyl glycol & Ethylhexylglycerin.
6. Homogenise the bulk for 2 minutes or until the desired viscosity is reached.

Additional details are provided below for some commercially available materials that are sold under the identified trade marks.

### Table of Commercially Available Materials

| | Trade | Active | % active | Active | |
|---|---|---|---|---|---|
| Supplier | name | INCI | INCI | compound | Excipients |
| Mibelle | Lipobelle ™ | Soy Isoflavones | | Genistein | Alcohol, lecithin, |
| | Soyaglycone | | | | Polsorbate 80 |
| | | | | | &Aqua. |
| DSM | Regu ®-Fade | Resveratrol | 100% | Trans resveratrol | None |
| Lipotec | Lipochroman ™ | Dimethylmethoxy | 100% | Dimethylmethoxy | None |
| | Molecule | chromanol | | chromanol | |

Table of commercially available Plant Extracts

| | | | % | | | |
|---|---|---|---|---|---|---|
| | | Active | active | Active | | Extraction |
| Supplier | Trade name | INCI | INCI | compound | Excipients | solvent |
| Indena/ | Centevita ™ | Centella | 100% | Asiatic and | None | None |
| Givaudan | | asiatica leaf | | madecassic acid | | |
| | | extract | | 45 to 100% | | |
| Croda | Phytessence ™ | Quercus | 5-10% | Polyphenols | Water 50-70%, | Ethanol/water |
| | French Oak | Petraea fruit | | 0.70-1.10% | Glycerin 25- | |
| | | extract | | | 50%, Sodium | |
| | | | | | benzoate approx | |
| | | | | | 0.6%, Citric | |
| | | | | | acid approx | |
| | | | | | 0.04% | |
| Merck | Emblica ® | Phyllanthus | 100% | Contains min. | 100% pure | |
| | | Emblica | | 60% low | | |
| | | fruit Extract | | molecular weight | | |
| | | | | hydrolysable | | |
| | | | | tannins, | | |
| | | | | Emblicanin A & | | |
| | | | | Emblicanin B | | |
| | | | | being the key | | |
| | | | | bio-active | | |
| | | | | ingredients | | |
| Clariant | Red Snow ® | Camellia | 1-5% | Protocatechuic | Propanediol 30- | Propanediol/ |
| | | Japonica | | acid min. | 70%, Aqua 30- | water |
| | | flower | | 200 ppm | 70% | |
| | | extract | | | | |
| Naturex | Botany | Ginkgo | 100% | Ginkgoflavon- | 100% pure | Hydroalcoholic |
| | Ginkgo ™ | Biloba leaf | | glucosides 24- | | |
| | | extract | | 30%, Terpene | | |
| | | | | lactones 6-9% | | |
| Silab | Desoxine ® | Myrtus | 2.65% | polyphenols | water 97.05%, | |
| | Bio | Communis | | 2.5 g/l-5.0 g/l | Sodium | |
| | | leaf extract | | | benzoate 0.3% | |
| BASF | Arganyl ® LS | Argania | Approx. | Flavonoides | Glycerin 47.00%, | Glycerin/ |
| | 9781 | Spinosa leaf | 5.00% | 0.30-0.45% | Phenoxyethanol | water |
| | | extract | | | 2.00%, Water to | |
| | | | | | 100% | |
| Horphag | Pycnogenol ® | Pinus | 100% | Procyanidins | 100% pure | |
| Research | | Pinaster | | 65-75% | | |
| | | back extract | | | | |
| Naturex | Green Tea | Camellia | 100% | Polyphenols >95%/ | 100% pure | n/a |
| | PE ™ | sinensis leaf | | Catechins >70% | | |
| | | extract | | | | |
| Silab | Cohesium ® | Ophiopogon | 9-13% | Sugars | Water | Water |
| | | japonicus | | (Fructose): 85- | | |
| | | root extract | | 127 g/L | | |

1. A cosmetic composition kit comprising at least two compositions, the first composition comprising:
a. Retinoid
b. Extract of Centella asiatica
and the second composition comprising at least one ingredient selected from the group consisting of ceramide, vitamin B, vitamin B derivative, extract of Ophiopogon japonicus and mixtures thereof.

2. A kit according to claim 1 wherein the first composition retinoid is selected from the group consisting of retinol, retinol isomers, retinaldehyde, retinaldehyde isomers and mixtures thereof.

3. A kit according to claim 1 wherein the first composition retinoid is microencapsulated.

4. A kit according to claim 1 wherein the first composition retinoid is microencapsulated with a material selected from the group consisting of sugar based emulsifier, co-emulsifier, a positively charged molecule, solvent and mixtures thereof.

5. A kit according to claim 1 wherein the first composition retinoid is present at a level of at least 0.5% by weight of the composition.

6. A kit according to claim 1 wherein the first composition extract of Centella asiatica is prepared from the leaf of the plant.

7. A kit according to claim 1 wherein the first composition extract of Centella asiatica comprises madecassoside.

8. A kit according to claim 1 wherein the first composition extract of Centella asiatica is present at a level of from 0.001 to 0.5% by weight of the composition.

9. A kit according to claim 1 wherein the second composition comprises at least two ingredients selected from the group consisting of ceramide, vitamin B, vitamin B derivative, extract of Ophiopogon japonicus and mixtures thereof.

10. A kit according to claim 1 wherein the second composition comprises ceramide, vitamin B and/or vitamin B derivative, extract of Ophiopogon japonicus and mixtures thereof.

11. A kit according to claim 1 wherein the second composition ceramide is selected from the group consisting of ceramide 1 (ceramide EOP), ceramide 2 (ceramide NG), ceramide 3 (ceramide NP), ceramide 6 (ceramide AP) and mixtures thereof.

12. A kit according to claim 1 A kit according to any preceding claim wherein the second composition ceramide is ceramide 6 (ceramide AP).

13. A kit according to claim 1 wherein the second composition ceramide is ceramide 3 (ceramide NP), also known as N-oloeoyl-4-hydroxysphinganine.

14. A kit according to claim 1 wherein the second composition vitamin B is selected from the group consisting of vitamin B1, vitamin B2, Vitamin B3, vitamin B4, vitamin B5, vitamin B6, vitamin B7, vitamin B8, vitamin B9, vitamin B10, vitamin B11, vitamin B12 and mixtures thereof.

15. A kit according to claim 1 wherein the second composition comprises vitamin B and said vitamin B is niacinamide.

16. A kit according to claim 1 wherein the second composition extract of Ophiopogon Japonicus and said extract is achieved from the root of the plant.

17. A process of application of cosmetic compositions to skin by providing the cosmetic composition kit of claim 1, first applying the first composition and subsequently applying the second composition in more or more applications in one or more application events.


SEQUENCE LISTING (51 sequences)

SEQ ID NO: 1 - protein, 4 aa, synthetic construct
GQPR

SEQ ID NO: 2 - protein, 4 aa, synthetic construct
AQTR

SEQ ID NO: 3 - protein, 4 aa, synthetic construct
RSRK

SEQ ID NO: 4 - protein, 4 aa, synthetic construct
QTAV

SEQ ID NO: 5 - protein, 4 aa, synthetic construct
EKGD

SEQ ID NO: 6 - protein, 4 aa, synthetic construct
ELGD

SEQ ID NO: 7 - protein, 4 aa, synthetic construct
EAGD

SEQ ID NO: 8 - protein, 4 aa, synthetic construct
EIGD

SEQ ID NO: 9 - protein, 4 aa, synthetic construct
ERGD

SEQ ID NO: 10 - protein, 4 aa, synthetic construct
KEGD

SEQ ID NO: 11 - protein, 4 aa, synthetic construct
KLGD

SEQ ID NO: 12 - protein, 4 aa, synthetic construct
KAGD

SEQ ID NO: 13 - protein, 4 aa, synthetic construct
KIGD

SEQ ID NO: 14 - protein, 4 aa, synthetic construct
KRGD

SEQ ID NO: 15 - protein, 4 aa, synthetic construct
LEGD

SEQ ID NO: 16 - protein, 4 aa, synthetic construct
LKGD

SEQ ID NO: 17 - protein, 4 aa, synthetic construct
LAGD

SEQ ID NO: 18 - protein, 4 aa, synthetic construct
LIGD

SEQ ID NO: 19 - protein, 4 aa, synthetic construct
LRGD

SEQ ID NO: 20 - protein, 4 aa, synthetic construct
IEGD

SEQ ID NO: 21 - protein, 4 aa, synthetic construct
IKGD

SEQ ID NO: 22 - protein, 4 aa, synthetic construct
ILGD

SEQ ID NO: 23 - protein, 4 aa, synthetic construct
IAGD

SEQ ID NO: 24 - protein, 4 aa, synthetic construct
IRGD

SEQ ID NO: 25 - protein, 4 aa, synthetic construct
REGD

SEQ ID NO: 26 - protein, 4 aa, synthetic construct
RKGD

SEQ ID NO: 27 - protein, 4 aa, synthetic construct
RLGD

SEQ ID NO: 28 - protein, 4 aa, synthetic construct
RAGD

SEQ ID NO: 29 - protein, 4 aa, synthetic construct
RIGD

SEQ ID NO: 30 - protein, 4 aa, synthetic construct
AEGD

SEQ ID NO: 31 - protein, 4 aa, synthetic construct
AKGD

SEQ ID NO: 32 - protein, 4 aa, synthetic construct
ALGD

SEQ ID NO: 33 - protein, 4 aa, synthetic construct
AIGD

SEQ ID NO: 34 - protein, 4 aa, synthetic construct
ARGD

SEQ ID NO: 35 - protein, 4 aa, synthetic construct
LSVD

SEQ ID NO: 36 - protein, 4 aa, synthetic construct
LSVP

SEQ ID NO: 37 - protein, 4 aa, synthetic construct
LSVG

SEQ ID NO: 38 - protein, 4 aa, synthetic construct
LSDV

SEQ ID NO: 39 - protein, 4 aa, synthetic construct
LSDP

SEQ ID NO: 40 - protein, 4 aa, synthetic construct
LSDG

SEQ ID NO: 41 - protein, 4 aa, synthetic construct
LSPV

SEQ ID NO: 42 - protein, 4 aa, synthetic construct
LSPD

SEQ ID NO: 43 - protein, 4 aa, synthetic construct
LSPG

SEQ ID NO: 44 - protein, 4 aa, synthetic construct
LSGV

SEQ ID NO: 45 - protein, 4 aa, synthetic construct
LSGD

SEQ ID NO: 46 - protein, 4 aa, synthetic construct
LSGP

SEQ ID NO: 47 - protein, 4 aa, synthetic construct
GPKG

SEQ ID NO: 48 - protein, 4 aa, synthetic construct
GPEG

SEQ ID NO: 49 - protein, 4 aa, synthetic construct
GPSG

SEQ ID NO: 50 - protein, 5 aa, synthetic construct
KTTKS

SEQ ID NO: 51 - protein, 5 aa, synthetic construct
RDKYV